SEQUENCE LISTING (33 sequences)

SEQ ID NO: 1 - protein, 119 aa, Artificial Sequence
  Synthetic Peptide
QSLEESGGDLVKPEGSLTLTCTASGFSFSTDYWICWVRQAPGKGLEWIACIYVGTSGDTYYATWAKGRFTISKTSSTTVTLQMTSLTAADTATYFCAGADNPYDYFNLWGPGTLVTVSS

SEQ ID NO: 2 - protein, 6 aa, Artificial Sequence
  Synthetic Peptide
TDYWIC

SEQ ID NO: 3 - protein, 11 aa, Artificial Sequence
  Synthetic Peptide
GFSFSTDYWIC

SEQ ID NO: 4 - protein, 9 aa, Artificial Sequence
  Synthetic Peptide
IYVGTSGDT

SEQ ID NO: 5 - protein, 18 aa, Artificial Sequence
  Synthetic Peptide
CIYVGTSGDTYYATWAKG

SEQ ID NO: 6 - protein, 10 aa, Artificial Sequence
  Synthetic Peptide
ADNPYDYFNL

SEQ ID NO: 7 - protein, 13 aa, Artificial Sequence
  Synthetic Peptide
AGADNPYDYFNLW

SEQ ID NO: 8 - protein, 109 aa, Artificial Sequence
  Synthetic Peptide
GTPDMTQTPASMEVAVGGTVTIKCQASETISGYLSWYQQKPGQRPKLLMYRASTLASGVSSRFKGSGSGTQFTLTISGVECADAATYYCQQGYSYSDTDNVFGGGTEVV

SEQ ID NO: 9 - protein, 6 aa, Artificial Sequence
  Synthetic Peptide
ETISGY

SEQ ID NO: 10 - protein, 11 aa, Artificial Sequence
  Synthetic Peptide
QASETISGYLS

SEQ ID NO: 11 - protein, 3 aa, Artificial Sequence
  Synthetic Peptide
RAS

SEQ ID NO: 12 - protein, 7 aa, Artificial Sequence
  Synthetic Peptide
RASTLAS

SEQ ID NO: 13 - protein, 7 aa, Artificial Sequence
  Synthetic Peptide
QQGYSYS

SEQ ID NO: 14 - protein, 12 aa, Artificial Sequence
  Synthetic Peptide
QQGYSYSDTDNV

SEQ ID NO: 15 - protein, 121 aa, Artificial Sequence
  Synthetic Peptide
QVQLQESGPGLVKPSDTLSLTCTVSGFSFSTDYWWSWIRQPPGKGLEWIGYIYVGTSGDTYYNPSLKSRVTISVDTSKNQFSLKLSSVTAADTAVYYCAGADNPYDYFNLWGQGTLVTVSS

SEQ ID NO: 16 - protein, 6 aa, Artificial Sequence
  Synthetic Peptide
TDYWWS

SEQ ID NO: 17 - protein, 10 aa, Artificial Sequence
  Synthetic Peptide
GFSFSTDYWW

SEQ ID NO: 18 - protein, 11 aa, Artificial Sequence
  Synthetic Peptide
GFSFSTDYWWS

SEQ ID NO: 19 - protein, 18 aa, Artificial Sequence
  Synthetic Peptide
YIYVGTSGDTYYNPSLKS

SEQ ID NO: 20 - protein, 110 aa, Artificial Sequence
  Synthetic Peptide
DIQMTQSPSSLSASVGDRVTITCRASETISGYLNWYQQKPGKAPKLLIYRASSLQSGVPSRFSGSGSGTDFTLTISSLQPEDFATYYCQQGYSYSDTDNVFGGGTKVEIK

SEQ ID NO: 21 - protein, 11 aa, Artificial Sequence
  Synthetic Peptide
RASETISGYLN

SEQ ID NO: 22 - protein, 7 aa, Artificial Sequence
  Synthetic Peptide
RASSLQS

SEQ ID NO: 23 - protein, 110 aa, Artificial Sequence
  Synthetic Peptide
DIQMTQSPSSLSASVGDRVTITCRASETISGYLAWYQQKPGKVPKLLIYRASTLQSGVPSRFSGSGSGTDFTLTISSLQPEDVATYYCQQGYSYSDTDNVFGGGTKVEIK

SEQ ID NO: 24 - protein, 11 aa, Artificial Sequence
  Synthetic Peptide
RASETISGYLA

SEQ ID NO: 25 - protein, 7 aa, Artificial Sequence
  Synthetic Peptide
RASTLQS

SEQ ID NO: 26 - protein, 120 aa, Artificial Sequence
  Synthetic Peptide
EVQLVESGGGLVQPGGSLRLSCAASGFSFSTDYWMSWVRQAPGKGLEWVSSIYVGTSGDTYYADSVKGRFTISRDNSKNTLYLQMDSLRAEDTAIYYCAGADNPYDYFNLWGQGTLVTVS

SEQ ID NO: 27 - protein, 6 aa, Artificial Sequence
  Synthetic Peptide
TDYWMS

SEQ ID NO: 28 - protein, 10 aa, Artificial Sequence
  Synthetic Peptide
GFSFSTDYWM

SEQ ID NO: 29 - protein, 11 aa, Artificial Sequence
  Synthetic Peptide
GFSFSTDYWMS

SEQ ID NO: 30 - protein, 18 aa, Artificial Sequence
  Synthetic Peptide
SIYVGTSGDTYYADSVKG

SEQ ID NO: 31 - protein, 110 aa, Artificial Sequence
  Synthetic Peptide
DIQMTQSPSSVSASVGDRVTITCRASETISGYLAWYQQKPGKAPKLLIYRASSLESGVPSRFSGSGSGTEFTLTISSLQPDDFATYYCQQGYSYSDTDNVFGGGTKVEIK

SEQ ID NO: 32 - protein, 7 aa, Artificial Sequence
  Synthetic Peptide
RASSLES

SEQ ID NO: 33 - protein, 8 aa, Artificial Sequence
  Synthetic Peptide
GFSFSTDY

(19) United States
(12) Patent Application Publication
(10) Pub. No.: US 2023/0050773 A1
(43) Pub. Date: Feb. 16, 2023

(54) ANTIBODIES AGAINST LIF AND USES THEREOF

(71) Applicant: MEDIMMUNE LIMITED, Cambridge (GB)

(72) Inventors: Arif JETHA, Toronto (CA); Johan FRANSSON, Toronto (CA)

(21) Appl. No.: 17/756,820

(22) PCT Filed: Dec. 3, 2020
(86) PCT No.: PCT/EP2020/084538

§ 371 Date: Jun. 3, 2022

Related U.S. Application Data
(60) Provisional application No. 62/943,479, filed on Dec. 4, 2019.

(51) Int. Cl. C07K 16/24; C12N 15/62; C12N 5/00; C12P 21/00

(52) U.S. Cl. CPC C07K 16/244; C12N 15/625; C12N 5/00; C12P 21/005; C07K 2317/24; C07K 2317/622; C07K 2317/569

(57) ABSTRACT

Disclosed herein are antibodies that specifically bind and inhibit the action of the IL-6 family member LIF that are useful in the treatment of cancer. Also disclosed herein are uses of said antibodies for the treatment of cancer.

### CROSS-REFERENCE TO RELATED APPLICATIONS

This application claims priority to U.S. provisional application 62/943,479, filed Dec. 4, 2019, which is hereby incorporated by reference in its entirety for all purposes.

### REFERENCE TO THE SEQUENCE LISTING

This application includes a Sequence Listing submitted electronically entitled “LIF-104-WO-PCT_ST25”, created on Nov. 24, 2020, and having a size of 12,982 bytes, which is hereby incorporated by reference in its entirety

### BACKGROUND

Leukemia inhibitory factor (LIF) is a pleiotropic cytokine that binds to the LIF receptor and activates signaling pathways that enhance cell proliferation and survival. High LIF expression correlates with negative outcome in several solid tumours, and it is hypothesized that blocking LIF binding to its receptor may improve disease outcome.

### SUMMARY

Described herein are antibodies that block the interaction of LIF with its receptor. These antibodies are useful in methods of treating individuals with cancer.

In one aspect, described herein, is a recombinant antibody that specifically binds Leukemia Inhibitory Factor (LIF) comprising: (a) a heavy chain complementarity determining region 1 (VH-CDR1) comprising the amino acid sequence set forth in any one of SEQ ID NOs: 2 (TDYWIC), 3 (GFSFSTDYWIC); or 33 (GFSFSTDY); (b) a heavy chain complementarity determining region 2 (VH-CDR2) comprising the amino acid sequence set forth in any one of SEQ ID NOs: 4 (IYVGTSGDT) or 5 (CIYVGTSGDTYYATWAKG); (c) a heavy chain complementarity determining region 3 (VH-CDR3) comprising the amino acid sequence set forth in any one of SEQ ID NOs: 6 (ADNPYDYFNL) or 7 (AGADNPYDYFNLW); (d) a light chain complementarity determining region 1 (VL-CDR1) comprising the amino acid sequence set forth in any one of SEQ ID NOs: 9 (ETISGY) or 10 (QASETISGYLS); (e) a light chain complementarity determining region 2 (VL-CDR2) comprising the amino acid sequence set forth in any one of SEQ ID NOs: 11 (RAS) or 12 (RASTLAS); and (f) a light chain complementarity determining region 3 (VL-CDR3) comprising the amino acid sequence set forth in any one of SEQ ID NOs: 13 (QQGYSYS) or 14 (QQGYSYSDTDNV). In certain embodiments, the recombinant antibody is chimeric or humanized. In certain embodiments, the recombinant antibody is an IgG antibody. In certain embodiments, the recombinant antibody comprises a Fab, F(ab)₂, a single-domain antibody, a single chain variable fragment (scFv), or a nanobody. In certain embodiments, described herein is a pharmaceutical composition comprising the recombinant antibody and a pharmaceutically acceptable excipient, carrier, or diluent. In certain embodiments, the pharmaceutical composition is formulated for intravenous administration. In certain embodiments, the recombinant antibody or the pharmaceutical composition is for use in treating cancer. In certain embodiments, the cancer comprises glioblastoma, pancreatic cancer, ovarian cancer, colon cancer, prostate cancer, or lung cancer. Also described is a method of treating a cancer in an individual comprising administering an effective amount of the recombinant antibody or the pharmaceutical composition to an individual diagnosed with or suspected of being afflicted with cancer. In certain embodiments, the cancer comprises glioblastoma, pancreatic cancer, ovarian cancer, colon cancer, prostate cancer, or lung cancer. Also contemplated is a nucleic acid encoding the recombinant antibody, and/or a cell comprising the nucleic acid. In certain embodiments, the cell is a eukaryotic cell. In certain embodiments, the cell is a Chinese Hamster Ovary (CHO) cell. Also contemplated, is a method of making a cancer treatment comprising incubating the cell in a culture medium under conditions sufficient to secrete the recombinant antibody into the culture medium. In certain embodiments, the method further comprises subjecting the culture medium to at least one purification step. Also contemplated, is a method of making a cancer treatment comprising admixing the recombinant antibody and a pharmaceutically acceptable excipient, carrier, or diluent.

In another aspect, described herein, is a recombinant antibody that specifically binds Leukemia Inhibitory Factor (LIF) comprising: (a) a heavy chain complementarity determining region 1 (VH-CDR1) comprising the amino acid sequence set forth in any one of SEQ ID NOs: 16 (TDYWWS); 17 (GFSFSTDYWW); or 18 (GFSFSTDYWWS); (b) a heavy chain complementarity determining region 2 (VH-CDR2) comprising the amino acid sequence set forth in any one of SEQ ID NOs: 4 (IYVGTSGDT) or 19 (YIYVGTSGDTYYNPSLKS); (c) a heavy chain complementarity determining region 3 (VH-CDR3) comprising the amino acid sequence set forth in any one of SEQ ID NOs: 6 (ADNPYDYFNL) or 7 (AGADNPYDYFNLW); (d) a light chain complementarity determining region 1 (VL-CDR1) comprising the amino acid sequence set forth in any one of SEQ ID NOs: 9 (ETISGY) or 21 (RASETISGYLN); (e) a light chain complementarity determining region 2 (VL-CDR2) comprising the amino acid sequence set forth in any one of SEQ ID NOs: 11 (RAS) or 22 (RASSLQS); and (f) a light chain complementarity determining region 3 (VL-CDR3) comprising the amino acid sequence set forth in any one of SEQ ID NOs: 13 (QQGYSYS) or 14 (QQGYSYSDTDNV). In certain embodiments, the recombinant antibody is chimeric or humanized. In certain embodiments, the recombinant antibody is an IgG antibody. In certain embodiments, the recombinant antibody comprises a Fab, F(ab)₂, a single-domain antibody, a single chain variable fragment (scFv), or a nanobody. In certain embodiments, described herein is a pharmaceutical composition comprising the recombinant antibody and a pharmaceutically acceptable excipient, carrier, or diluent. In certain embodiments, the pharmaceutical composition is formulated for intravenous administration. In certain embodiments, the recombinant antibody or the pharmaceutical composition is for use in treating cancer. In certain embodiments, the cancer comprises glioblastoma, pancreatic cancer, ovarian cancer, colon cancer, prostate cancer, or lung cancer. Also described is a method of treating a cancer in an individual comprising administering an effective amount of the recombinant antibody or the pharmaceutical composition to an individual diagnosed with or suspected of being afflicted with cancer. In certain embodiments, the cancer comprises glioblastoma, pancreatic cancer, ovarian cancer, colon cancer, prostate cancer, or lung cancer. Also contemplated is a nucleic acid encoding the recombinant antibody, and/or a cell comprising the nucleic acid. In certain embodiments, the cell is a eukaryotic cell. In certain embodiments, the cell is a Chinese Hamster Ovary (CHO) cell. Also contemplated, is a method of making a cancer treatment comprising incubating the cell in a culture medium under conditions sufficient to secrete the recombinant antibody into the culture medium. In certain embodiments, the method further comprises subjecting the culture medium to at least one purification step. Also contemplated, is a method of making a cancer treatment comprising admixing the recombinant antibody and a pharmaceutically acceptable excipient, carrier, or diluent.

In another aspect, described herein, is a recombinant antibody that specifically binds Leukemia Inhibitory Factor (LIF) comprising: (a) a heavy chain complementarity determining region 1 (VH-CDR1) comprising the amino acid sequence set forth in any one of SEQ ID NOs: 16 (TDYWWS); 17 (GFSFSTDYWW); or 18 (GFSFSTDYWWS); (b) a heavy chain complementarity determining region 2 (VH-CDR2) comprising the amino acid sequence set forth in any one of SEQ ID NOs: 4 (IYVGTSGDT) or 19 (YIYVGTSGDTYYNPSLKS); (c) a heavy chain complementarity determining region 3 (VH-CDR3) comprising the amino acid sequence set forth in any one of SEQ ID NOs: 6 (ADNPYDYFNL) or 7 (AGADNPYDYFNLW); (d) a light chain complementarity determining region 1 (VL-CDR1) comprising the amino acid sequence set forth in any one of SEQ ID NOs: 9 (ETISGY) or 24 (RASETISGYLA); (e) a light chain complementarity determining region 2 (VL-CDR2) comprising the amino acid sequence set forth in any one of SEQ ID NOs: 11 (RAS) or 25 (RASTLQS); and (f) a light chain complementarity determining region 3 (VL-CDR3) comprising the amino acid sequence set forth in any one of SEQ ID NOs: 13 (QQGYSYS) or 14 (QQGYSYSDTDNV). In certain embodiments, the recombinant antibody is chimeric or humanized. In certain embodiments, the recombinant antibody is an IgG antibody. In certain embodiments, the recombinant antibody comprises a Fab, F(ab)₂, a single-domain antibody, a single chain variable fragment (scFv), or a nanobody. In certain embodiments, described herein is a pharmaceutical composition comprising the recombinant antibody and a pharmaceutically acceptable excipient, carrier, or diluent. In certain embodiments, the pharmaceutical composition is formulated for intravenous administration. In certain embodiments, the recombinant antibody or the pharmaceutical composition is for use in treating cancer. In certain embodiments, the cancer comprises glioblastoma, pancreatic cancer, ovarian cancer, colon cancer, prostate cancer, or lung cancer. Also described is a method of treating a cancer in an individual comprising administering an effective amount of the recombinant antibody or the pharmaceutical composition to an individual diagnosed with or suspected of being afflicted with cancer. In certain embodiments, the cancer comprises glioblastoma, pancreatic cancer, ovarian cancer, colon cancer, prostate cancer, or lung cancer. Also contemplated is a nucleic acid encoding the recombinant antibody, and/or a cell comprising the nucleic acid. In certain embodiments, the cell is a eukaryotic cell. In certain embodiments, the cell is a Chinese Hamster Ovary (CHO) cell. Also contemplated, is a method of making a cancer treatment comprising incubating the cell in a culture medium under conditions sufficient to secrete the recombinant antibody into the culture medium. In certain embodiments, the method further comprises subjecting the culture medium to at least one purification step. Also contemplated, is a method of making a cancer treatment comprising admixing the recombinant antibody and a pharmaceutically acceptable excipient, carrier, or diluent.

In another aspect, described herein, is a recombinant antibody that specifically binds Leukemia Inhibitory Factor (LIF) comprising: (a) a heavy chain complementarity determining region 1 (VH-CDR1) comprising the amino acid sequence set forth in any one of SEQ ID NOs: 27 (TDYWMS); 28 (GFSFSTDYWM); or 29 (GFSFSTDYWMS); (b) a heavy chain complementarity determining region 2 (VH-CDR2) comprising the amino acid sequence set forth in any one of SEQ ID NOs: 4 (IYVGTSGDT) or 30 (SIYVGTSGDTYYADSVKG); (c) a heavy chain complementarity determining region 3 (VH-CDR3) comprising the amino acid sequence set forth in any one of SEQ ID NOs: 6 (ADNPYDYFNL) or 7 (AGADNPYDYFNLW); (d) a light chain complementarity determining region 1 (VL-CDR1) comprising the amino acid sequence set forth in any one of SEQ ID NOs: 9 (ETISGY) or 24 (RASETISGYLA); (e) a light chain complementarity determining region 2 (VL-CDR2) comprising the amino acid sequence set forth in any one of SEQ ID NOs: 11 (RAS) or 32 (RASSLES); and (f) a light chain complementarity determining region 3 (VL-CDR3) comprising the amino acid sequence set forth in any one of SEQ ID NOs: 13 (QQGYSYS) or 14 (QQGYSYSDTDNV). In certain embodiments, the recombinant antibody is chimeric or humanized. In certain embodiments, the recombinant antibody is an IgG antibody. In certain embodiments, the recombinant antibody comprises a Fab, F(ab)₂, a single-domain antibody, a single chain variable fragment (scFv), or a nanobody. In certain embodiments, described herein is a pharmaceutical composition comprising the recombinant antibody and a pharmaceutically acceptable excipient, carrier, or diluent. In certain embodiments, the pharmaceutical composition is formulated for intravenous administration. In certain embodiments, the recombinant antibody or the pharmaceutical composition is for use in treating cancer. In certain embodiments, the cancer comprises glioblastoma, pancreatic cancer, ovarian cancer, colon cancer, prostate cancer, or lung cancer. Also described is a method of treating a cancer in an individual comprising administering an effective amount of the recombinant antibody or the pharmaceutical composition to an individual diagnosed with or suspected of being afflicted with cancer. In certain embodiments, the cancer comprises glioblastoma, pancreatic cancer, ovarian cancer, colon cancer, prostate cancer, or lung cancer. Also contemplated is a nucleic acid encoding the recombinant antibody, and/or a cell comprising the nucleic acid. In certain embodiments, the cell is a eukaryotic cell. In certain embodiments, the cell is a Chinese Hamster Ovary (CHO) cell. Also contemplated, is a method of making a cancer treatment comprising incubating the cell in a culture medium under conditions sufficient to secrete the recombinant antibody into the culture medium. In certain embodiments, the method further comprises subjecting the culture medium to at least one purification step. Also contemplated, is a method of making a cancer treatment comprising admixing the recombinant antibody and a pharmaceutically acceptable excipient, carrier, or diluent.

In another aspect, described herein, is a recombinant antibody that specifically binds Leukemia Inhibitory Factor (LIF) comprising: (a) a heavy chain complementarity determining region 1 (VH-CDR1) comprising the amino acid sequence set forth in any one of SEQ ID NOs: 27 (TDYWMS); 28 (GFSFSTDYWM); or 29 (GFSFSTDYWMS); (b) a heavy chain complementarity determining region 2 (VH-CDR2) comprising the amino acid sequence set forth in any one of SEQ ID NOs: 4 (IYVGTSGDT) or 30 (SIYVGTSGDTYYADSVKG); (c) a heavy chain complementarity determining region 3 (VH-CDR3) comprising the amino acid sequence set forth in any one of SEQ ID NOs: 6 (ADNPYDYFNL) or 7 (AGADNPYDYFNLW); (d) a light chain complementarity determining region 1 (VL-CDR1) comprising the amino acid sequence set forth in any one of SEQ ID NOs: 9 (ETISGY) or 21 (RASETISGYLN); (e) a light chain complementarity determining region 2 (VL-CDR2) comprising the amino acid sequence set forth in any one of SEQ ID NOs: 11 (RAS) or 22 (RASSLQS); and (f) a light chain complementarity determining region 3 (VL-CDR3) comprising the amino acid sequence set forth in any one of SEQ ID NOs: 13 (QQGYSYS) or 14 (QQGYSYSDTDNV). In certain embodiments, the recombinant antibody is chimeric or humanized. In certain embodiments, the recombinant antibody is an IgG antibody. In certain embodiments, the recombinant antibody comprises a Fab, F(ab)₂, a single-domain antibody, a single chain variable fragment (scFv), or a nanobody. In certain embodiments, described herein is a pharmaceutical composition comprising the recombinant antibody and a pharmaceutically acceptable excipient, carrier, or diluent. In certain embodiments, the pharmaceutical composition is formulated for intravenous administration. In certain embodiments, the recombinant antibody or the pharmaceutical composition is for use in treating cancer. In certain embodiments, the cancer comprises glioblastoma, pancreatic cancer, ovarian cancer, colon cancer, prostate cancer, or lung cancer. Also described is a method of treating a cancer in an individual comprising administering an effective amount of the recombinant antibody or the pharmaceutical composition to an individual diagnosed with or suspected of being afflicted with cancer. In certain embodiments, the cancer comprises glioblastoma, pancreatic cancer, ovarian cancer, colon cancer, prostate cancer, or lung cancer. Also contemplated is a nucleic acid encoding the recombinant antibody, and/or a cell comprising the nucleic acid. In certain embodiments, the cell is a eukaryotic cell. In certain embodiments, the cell is a Chinese Hamster Ovary (CHO) cell. Also contemplated, is a method of making a cancer treatment comprising incubating the cell in a culture medium under conditions sufficient to secrete the recombinant antibody into the culture medium. In certain embodiments, the method further comprises subjecting the culture medium to at least one purification step. Also contemplated, is a method of making a cancer treatment comprising admixing the recombinant antibody and a pharmaceutically acceptable excipient, carrier, or diluent.

In another aspect, described herein, is a recombinant antibody that specifically binds Leukemia Inhibitory Factor (LIF) comprising: (a) a heavy chain complementarity determining region 1 (VH-CDR1) comprising the amino acid sequence set forth in SEQ ID NO: 33 (TDYW); (b) a heavy chain complementarity determining region 2 (VH-CDR2) comprising the amino acid sequence set forth in an SEQ ID NO: 4 (IYVGTSGDT); (c) a heavy chain complementarity determining region 3 (VH-CDR3) comprising the amino acid sequence set forth in any one of SEQ ID NOs: 6 (ADNPYDYFNL) or 7 (AGADNPYDYFNLW); (d) a light chain complementarity determining region 1 (VL-CDR1) comprising the amino acid sequence set forth in SEQ ID NO: 9 (ETISGY); (e) a light chain complementarity determining region 2 (VL-CDR2) comprising the amino acid sequence set forth in SEQ ID NO: 11 (RAS); and (f) a light chain complementarity determining region 3 (VL-CDR3) comprising the amino acid sequence set forth in any one of SEQ ID NOs: 13 (QQGYSYS) or 14 (QQGYSYSDTDNV). In certain embodiments, the recombinant antibody is chimeric or humanized. In certain embodiments, the recombinant antibody is an IgG antibody. In certain embodiments, the recombinant antibody comprises a Fab, F(ab)₂, a single-domain antibody, a single chain variable fragment (scFv), or a nanobody. In certain embodiments, described herein is a pharmaceutical composition comprising the recombinant antibody and a pharmaceutically acceptable excipient, carrier, or diluent. In certain embodiments, the pharmaceutical composition is formulated for intravenous administration. In certain embodiments, the recombinant antibody or the pharmaceutical composition is for use in treating cancer. In certain embodiments, the cancer comprises glioblastoma, pancreatic cancer, ovarian cancer, colon cancer, prostate cancer, or lung cancer. Also described is a method of treating a cancer in an individual comprising administering an effective amount of the recombinant antibody or the pharmaceutical composition to an individual diagnosed with or suspected of being afflicted with cancer. In certain embodiments, the cancer comprises glioblastoma, pancreatic cancer, ovarian cancer, colon cancer, prostate cancer, or lung cancer. Also contemplated is a nucleic acid encoding the recombinant antibody, and/or a cell comprising the nucleic acid. In certain embodiments, the cell is a eukaryotic cell. In certain embodiments, the cell is a Chinese Hamster Ovary (CHO) cell. Also contemplated, is a method of making a cancer treatment comprising incubating the cell in a culture medium under conditions sufficient to secrete the recombinant antibody into the culture medium. In certain embodiments, the method further comprises subjecting the culture medium to at least one purification step. Also contemplated, is a method of making a cancer treatment comprising admixing the recombinant antibody and a pharmaceutically acceptable excipient, carrier, or diluent.

In another aspect, described herein, is a recombinant antibody that specifically binds Leukemia Inhibitory Factor (LIF) comprising: an immunoglobulin heavy chain variable region (VH) sequence with an amino acid sequence at least about 85%, 90%, 95%, 97%, 98%, 99%, or 100% identical to the amino acid sequence set forth in SEQ ID NO: 1; and an immunoglobulin light chain variable region (VL) sequence with an amino acid sequence at least about 85%, 90%, 95%, 97%, 98%, 99%, or 100% identical to the amino acid sequence set forth in SEQ ID NO: 8. In certain embodiments, the recombinant antibody is chimeric or humanized. In certain embodiments, the recombinant antibody is an IgG antibody. In certain embodiments, the recombinant antibody comprises a Fab, F(ab)₂, a single-domain antibody, a single chain variable fragment (scFv), or a nanobody.

In another aspect, described herein, is a recombinant antibody that specifically binds Leukemia Inhibitory Factor (LIF) comprising: an immunoglobulin heavy chain variable region (VH) sequence with an amino acid sequence at least about 85%, 90%, 95%, 97%, 98%, 99%, or 100% identical to the amino acid sequence set forth in SEQ ID NO: 15; and an immunoglobulin light chain variable region (VL) sequence with an amino acid sequence at least about 85%, 90%, 95%, 97%, 98%, 99%, or 100% identical to the amino acid sequence set forth in SEQ ID NO: 20. In certain embodiments, the recombinant antibody is chimeric or humanized. In certain embodiments, the recombinant antibody is an IgG antibody. In certain embodiments, the recombinant antibody comprises a Fab, F(ab)₂, a single-domain antibody, a single chain variable fragment (scFv), or a nanobody. In certain embodiments, described herein is a pharmaceutical composition comprising the recombinant antibody and a pharmaceutically acceptable excipient, carrier, or diluent. In certain embodiments, the pharmaceutical composition is formulated for intravenous administration. In certain embodiments, the recombinant antibody or the pharmaceutical composition is for use in treating cancer. In certain embodiments, the cancer comprises glioblastoma, pancreatic cancer, ovarian cancer, colon cancer, prostate cancer, or lung cancer. Also described is a method of treating a cancer in an individual comprising administering an effective amount of the recombinant antibody or the pharmaceutical composition to an individual diagnosed with or suspected of being afflicted with cancer. In certain embodiments, the cancer comprises glioblastoma, pancreatic cancer, ovarian cancer, colon cancer, prostate cancer, or lung cancer. Also contemplated is a nucleic acid encoding the recombinant antibody, and/or a cell comprising the nucleic acid. In certain embodiments, the cell is a eukaryotic cell. In certain embodiments, the cell is a Chinese Hamster Ovary (CHO) cell. Also contemplated, is a method of making a cancer treatment comprising incubating the cell in a culture medium under conditions sufficient to secrete the recombinant antibody into the culture medium. In certain embodiments, the method further comprises subjecting the culture medium to at least one purification step. Also contemplated, is a method of making a cancer treatment comprising admixing the recombinant antibody and a pharmaceutically acceptable excipient, carrier, or diluent.

In another aspect, described herein, is a recombinant antibody that specifically binds Leukemia Inhibitory Factor (LIF) comprising: an immunoglobulin heavy chain variable region (VH) sequence with an amino acid sequence at least about 85%, 90%, 95%, 97%, 98%, 99%, or 100% identical to the amino acid sequence set forth in SEQ ID NO: 15; and an immunoglobulin light chain variable region (VL) sequence with an amino acid sequence at least about 85%, 90%, 95%, 97%, 98%, 99%, or 100% identical to the amino acid sequence set forth in SEQ ID NO: 23. In certain embodiments, the recombinant antibody is chimeric or humanized. In certain embodiments, the recombinant antibody is an IgG antibody. In certain embodiments, the recombinant antibody comprises a Fab, F(ab)₂, a single-domain antibody, a single chain variable fragment (scFv), or a nanobody. In certain embodiments, described herein is a pharmaceutical composition comprising the recombinant antibody and a pharmaceutically acceptable excipient, carrier, or diluent. In certain embodiments, the pharmaceutical composition is formulated for intravenous administration. In certain embodiments, the recombinant antibody or the pharmaceutical composition is for use in treating cancer. In certain embodiments, the cancer comprises glioblastoma, pancreatic cancer, ovarian cancer, colon cancer, prostate cancer, or lung cancer. Also described is a method of treating a cancer in an individual comprising administering an effective amount of the recombinant antibody or the pharmaceutical composition to an individual diagnosed with or suspected of being afflicted with cancer. In certain embodiments, the cancer comprises glioblastoma, pancreatic cancer, ovarian cancer, colon cancer, prostate cancer, or lung cancer. Also contemplated is a nucleic acid encoding the recombinant antibody, and/or a cell comprising the nucleic acid. In certain embodiments, the cell is a eukaryotic cell. In certain embodiments, the cell is a Chinese Hamster Ovary (CHO) cell. Also contemplated, is a method of making a cancer treatment comprising incubating the cell in a culture medium under conditions sufficient to secrete the recombinant antibody into the culture medium. In certain embodiments, the method further comprises subjecting the culture medium to at least one purification step. Also contemplated, is a method of making a cancer treatment comprising admixing the recombinant antibody and a pharmaceutically acceptable excipient, carrier, or diluent.

In another aspect, described herein, is a recombinant antibody that specifically binds Leukemia Inhibitory Factor (LIF) comprising: an immunoglobulin heavy chain variable region (VH) sequence with an amino acid sequence at least about 85%, 90%, 95%, 97%, 98%, 99%, or 100% identical to the amino acid sequence set forth in SEQ ID NO: 26; and an immunoglobulin light chain variable region (VL) sequence with an amino acid sequence at least about 85%, 90%, 95%, 97%, 98%, 99%, or 100% identical to the amino acid sequence set forth in SEQ ID NO: 31. In certain embodiments, the recombinant antibody is chimeric or humanized. In certain embodiments, the recombinant antibody is an IgG antibody. In certain embodiments, the recombinant antibody comprises a Fab, F(ab)₂, a single-domain antibody, a single chain variable fragment (scFv), or a nanobody. In certain embodiments, described herein is a pharmaceutical composition comprising the recombinant antibody and a pharmaceutically acceptable excipient, carrier, or diluent. In certain embodiments, the pharmaceutical composition is formulated for intravenous administration. In certain embodiments, the recombinant antibody or the pharmaceutical composition is for use in treating cancer. In certain embodiments, the cancer comprises glioblastoma, pancreatic cancer, ovarian cancer, colon cancer, prostate cancer, or lung cancer. Also described is a method of treating a cancer in an individual comprising administering an effective amount of the recombinant antibody or the pharmaceutical composition to an individual diagnosed with or suspected of being afflicted with cancer. In certain embodiments, the cancer comprises glioblastoma, pancreatic cancer, ovarian cancer, colon cancer, prostate cancer, or lung cancer. Also contemplated is a nucleic acid encoding the recombinant antibody, and/or a cell comprising the nucleic acid. In certain embodiments, the cell is a eukaryotic cell. In certain embodiments, the cell is a Chinese Hamster Ovary (CHO) cell. Also contemplated, is a method of making a cancer treatment comprising incubating the cell in a culture medium under conditions sufficient to secrete the recombinant antibody into the culture medium. In certain embodiments, the method further comprises subjecting the culture medium to at least one purification step. Also contemplated, is a method of making a cancer treatment comprising admixing the recombinant antibody and a pharmaceutically acceptable excipient, carrier, or diluent.

In another aspect, described herein, is a recombinant antibody that specifically binds Leukemia Inhibitory Factor (LIF) comprising: an immunoglobulin heavy chain variable region (VH) sequence with an amino acid sequence at least about 85%, 90%, 95%, 97%, 98%, 99%, or 100% identical to the amino acid sequence set forth in SEQ ID NO: 26; and an immunoglobulin light chain variable region (VL) sequence with an amino acid sequence at least about 85%, 90%, 95%, 97%, 98%, 99%, or 100% identical to the amino acid sequence set forth in SEQ ID NO: 20. In certain embodiments, the recombinant antibody is chimeric or humanized. In certain embodiments, the recombinant antibody is an IgG antibody. In certain embodiments, the recombinant antibody comprises a Fab, F(ab)₂, a single-domain antibody, a single chain variable fragment (scFv), or a nanobody. In certain embodiments, described herein is a pharmaceutical composition comprising the recombinant antibody and a pharmaceutically acceptable excipient, carrier, or diluent. In certain embodiments, the pharmaceutical composition is formulated for intravenous administration. In certain embodiments, the recombinant antibody or the pharmaceutical composition is for use in treating cancer. In certain embodiments, the cancer comprises glioblastoma, pancreatic cancer, ovarian cancer, colon cancer, prostate cancer, or lung cancer. Also described is a method of treating a cancer in an individual comprising administering an effective amount of the recombinant antibody or the pharmaceutical composition to an individual diagnosed with or suspected of being afflicted with cancer. In certain embodiments, the cancer comprises glioblastoma, pancreatic cancer, ovarian cancer, colon cancer, prostate cancer, or lung cancer. Also contemplated is a nucleic acid encoding the recombinant antibody, and/or a cell comprising the nucleic acid. In certain embodiments, the cell is a eukaryotic cell. In certain embodiments, the cell is a Chinese Hamster Ovary (CHO) cell. Also contemplated, is a method of making a cancer treatment comprising incubating the cell in a culture medium under conditions sufficient to secrete the recombinant antibody into the culture medium. In certain embodiments, the method further comprises subjecting the culture medium to at least one purification step. Also contemplated, is a method of making a cancer treatment comprising admixing the recombinant antibody and a pharmaceutically acceptable excipient, carrier, or diluent.

### BRIEF DESCRIPTION OF THE DRAWINGS

FIG. 1 depicts a single point binding screen for 15 humanized NB0880 variants and selection of NB0919, NB0920, NB0937 and NB0938 for further study.

FIG. 2 depicts binding of chimeric mAb B7 and its humanized variants to recombinant human LIF.

FIG. 3 depicts blocking activity of chimeric mAb B7 and its humanized variants in a recombinant human LIF:LIFR binding assay.

FIG. 4 depicts inhibition of pSTAT3 signaling by chimeric mAb B7 and its humanized variants in a cell based functional assay.

### DETAILED DESCRIPTION

In the following description, certain specific details are set forth in order to provide a thorough understanding of various embodiments. However, one skilled in the art will understand that the embodiments provided may be practiced without these details. Unless the context requires otherwise, throughout the specification and claims which follow, the word “comprise” and variations thereof, such as, “comprises” and “comprising” are to be construed in an open, inclusive sense, that is, as “including, but not limited to.” As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. Further, headings provided herein are for convenience only and do not interpret the scope or meaning of the claimed embodiments.

As used herein the term “about” refers to an amount that is near the stated amount by 10% or less.

As used herein the term “individual,” “patient,” or “subject” refers to individuals diagnosed with, suspected of being afflicted with, or at-risk of developing at least one disease for which the described compositions and method are useful for treating. In certain embodiments, the individual is a mammal. In certain embodiments, the mammal is a mouse, rat, rabbit, dog, cat, horse, cow, sheep, pig, goat, llama, alpaca, or yak. In certain embodiments, the individual is a human.

Among the provided antibodies are monoclonal antibodies, polyclonal antibodies, multispecific antibodies (for example, bispecific antibodies and polyreactive antibodies), and antibody fragments. The antibodies include antibody-conjugates and molecules comprising the antibodies, such as chimeric molecules. Thus, an antibody includes, but is not limited to, full-length and native antibodies, as well as fragments and portion thereof retaining the binding specificities thereof, such as any specific binding portion thereof including those having any number of, immunoglobulin classes and/or isotypes (e.g., IgG1, IgG2, IgG3, IgG4, IgM, IgA, IgD, IgE and IgM); and biologically relevant (antigen-binding) fragments or specific binding portions thereof, including but not limited to Fab, F(ab′)2, Fv, and scFv (single chain or related entity). A monoclonal antibody is generally one within a composition of substantially homogeneous antibodies; thus, any individual antibodies comprised within the monoclonal antibody composition are identical except for possible naturally occurring mutations that may be present in minor amounts. A polyclonal antibody is a preparation that includes different antibodies of varying sequences that generally are directed against two or more different determinants (epitopes). The monoclonal antibody can comprise a human IgG1 constant region. The monoclonal antibody can comprise a human IgG4 constant region.

The term “antibody” herein is used in the broadest sense and includes polyclonal and monoclonal antibodies, including intact antibodies and functional (antigen-binding) antibody fragments thereof, including fragment antigen binding (Fab) fragments, F(ab′)2 fragments, Fab′ fragments, Fv fragments, recombinant IgG (rIgG) fragments, single chain antibody fragments, including single chain variable fragments (sFv or scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD. The antibody can comprise a human IgG1 constant region. The antibody can comprise a human IgG4 constant region.

The terms “complementarity determining region,” and “CDR,” which are synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4). The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme), Al-Lazikani et al., (1997) JMB 273,927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme); Honegger A and Plückthun A, “Yet another numbering scheme for immunoglobulin variable domains: an automatic modeling and analysis tool,” J Mol Biol, 2001 Jun. 8; 309(3):657-70, (“Aho” numbering scheme); and Whitelegg N R and Rees A R, “WAM: an improved algorithm for modelling antibodies on the WEB,” Protein Eng. 2000 December; 13(12):819-24 (“AbM” numbering scheme.

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme.

The term “variable region” or “variable domain” refers to the domain of an antibody heavy or light chain that is involved in binding the antibody to antigen. The variable domains of the heavy chain and light chain (V_{H} and V_{L}, respectively) of a native antibody generally have similar structures, with each domain comprising four conserved framework regions (FRs) and three CDRs (See e.g., Kindt et al. Kuby Immunology, 6th ed., W.H. Freeman and Co., page 91(2007)). A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively (See e.g., Portolano et al., J. Immunol. 150:880-887 (1993); Clarkson et al., Nature 352:624-628 (1991)).

Among the provided antibodies are antibody fragments. An “antibody fragment” refers to a molecule other than an intact antibody that comprises a portion of an intact antibody that binds the antigen to which the intact antibody binds. Examples of antibody fragments include, but are not limited to, Fv, Fab, Fab′, Fab′-SH, F(ab′)₂; diabodies; linear antibodies; single-chain antibody molecules (e.g. scFv or sFv); and multispecific antibodies formed from antibody fragments. In particular embodiments, the antibodies are single-chain antibody fragments comprising a variable heavy chain region and/or a variable light chain region, such as scFvs.

Antibody fragments can be made by various techniques, including but not limited to proteolytic digestion of an intact antibody as well as production by recombinant host cells. In some embodiments, the antibodies are recombinantly-produced fragments, such as fragments comprising arrangements that do not occur naturally, such as those with two or more antibody regions or chains joined by synthetic linkers, e.g., polypeptide linkers, and/or those that are not produced by enzyme digestion of a naturally-occurring intact antibody. In some aspects, the antibody fragments are scFvs.

A “humanized” antibody is an antibody in which all or substantially all CDR amino acid residues are derived from non-human CDRs and all or substantially all FR amino acid residues are derived from human FRs. A humanized antibody optionally may include at least a portion of an antibody constant region derived from a human antibody. A “humanized form” of a non-human antibody refers to a variant of the non-human antibody that has undergone humanization, typically to reduce immunogenicity to humans, while retaining the specificity and affinity of the parental non-human antibody. In some embodiments, some FR residues in a humanized antibody are substituted with corresponding residues from a non-human antibody (e.g., the antibody from which the CDR residues are derived), e.g., to restore or improve antibody specificity or affinity.

Among the provided antibodies are human antibodies. A “human antibody” is an antibody with an amino acid sequence corresponding to that of an antibody produced by a human or a human cell, or non-human source that utilizes human antibody repertoires or other human antibody-encoding sequences, including human antibody libraries. The term excludes humanized forms of non-human antibodies comprising non-human antigen-binding regions, such as those in which all or substantially all CDRs are non-human.

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic animals, the endogenous immunoglobulin loci have generally been inactivated. Human antibodies also may be derived from human antibody libraries, including phage display and cell-free libraries, containing antibody-encoding sequences derived from a human repertoire.

The terms “polypeptide” and “protein” are used interchangeably to refer to a polymer of amino acid residues, and are not limited to a minimum length. Polypeptides, including the provided antibodies and antibody chains and other peptides, e.g., linkers and binding peptides, may include amino acid residues including natural and/or non-natural amino acid residues. The terms also include post-expression modifications of the polypeptide, for example, glycosylation, sialylation, acetylation, phosphorylation, and the like. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

Percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

In some embodiments, amino acid sequence variants of the antibodies provided herein are contemplated. A variant typically differs from a polypeptide specifically disclosed herein in one or more substitutions, deletions, additions and/or insertions. Such variants can be naturally occurring or can be synthetically generated, for example, by modifying one or more of the above polypeptide sequences of the invention and evaluating one or more biological activities of the polypeptide as described herein and/or using any of a number of known techniques. For example, it may be desirable to improve the binding affinity and/or other biological properties of the antibody Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

In some embodiments, antibody variants having one or more amino acid substitutions are provided. Sites of interest for mutagenesis by substitution include the CDRs and FRs. Amino acid substitutions may be introduced into an antibody of interest and the products screened for a desired activity, e.g., retained/improved antigen binding, decreased immunogenicity, or improved ADCC or CDC.

In some embodiments, substitutions, insertions, or deletions may occur within one or more CDRs, wherein the substitutions, insertions, or deletions do not substantially reduce antibody binding to antigen. For example, conservative substitutions that do not substantially reduce binding affinity may be made in CDRs. Such alterations may be outside of CDR “hotspots”. In some embodiments of the variant V_{H} and V_{L} sequences, each CDR is unaltered.

Alterations (e.g., substitutions) may be made in CDRs, e.g., to improve antibody affinity. Such alterations may be made in CDR encoding codons with a high mutation rate during somatic maturation (See e.g., Chowdhury, Methods Mol. Biol. 207:179-196 (2008)), and the resulting variant can be tested for binding affinity. Affinity maturation (e.g., using error-prone PCR, chain shuffling, randomization of CDRs, or oligonucleotide-directed mutagenesis) can be used to improve antibody affinity (See e.g., Hoogenboom et al. in Methods in Molecular Biology 178:1-37 (2001)). CDR residues involved in antigen binding may be specifically identified, e.g., using alanine scanning mutagenesis or modeling (See e.g., Cunningham and Wells Science, 244:1081-1085 (1989)). CDR-H3 and CDR-L3 in particular are often targeted. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions and deletions include amino- and/or carboxyl-terminal fusions ranging in length from one residue to polypeptides containing a hundred or more residues, as well as intrasequence insertions and deletions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody. Examples of intrasequence insertion variants of the antibody molecules include an insertion of 3 amino acids in the light chain. Examples of terminal deletions include an antibody with a deletion of 7 or less amino acids at an end of the light chain.

In some embodiments, the antibodies are altered to increase or decrease their glycosylation (e.g., by altering the amino acid sequence such that one or more glycosylation sites are created or removed). A carbohydrate attached to an Fc region of an antibody may be altered. Native antibodies from mammalian cells typically comprise a branched, biantennary oligosaccharide attached by an N-linkage to Asn297 of the CH2 domain of the Fc region (See e.g., Wright et al. TIBTECH 15:26-32 (1997)). The oligosaccharide can be various carbohydrates, e.g., mannose, N-acetyl glucosamine (GlcNAc), galactose, sialic acid, fucose attached to a GlcNAc in the stem of the biantennar oligosaccharide structure. Modifications of the oligosaccharide in an antibody can be made, for example, to create antibody variants with certain improved properties. Antibody glycosylation variants can have improved ADCC and/or CDC function. In some embodiments, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn297 (See e.g., WO 08/077546). Asn297 refers to the asparagine residue located at about position 297 in the Fc region (EU numbering of Fc region residues; See e.g., Edelman et al. Proc Natl Acad Sci USA. 1969 May; 63(1):78-85). However, Asn297 may also be located about ±3 amino acids upstream or downstream of position 297, i.e., between positions 294 and 300, due to minor sequence variations in antibodies. Such fucosylation variants can have improved ADCC function (See e.g., Okazaki et al. J. Mol. Biol. 336:1239-1249 (2004); and Yamane-Ohnuki et al. Biotech. Bioeng. 87: 614 (2004)). Cell lines, e.g., knockout cell lines and methods of their use can be used to produce defucosylated antibodies, e.g., Lec13 CHO cells deficient in protein fucosylation and alpha-1,6-fucosyltransferase gene (FUT8) knockout CHO cells (See e.g., Ripka et al. Arch. Biochem. Biophys. 249:533-545 (1986); Yamane-Ohnuki et al. Biotech. Bioeng. 87: 614 (2004); Kanda, Y. et al., Biotechnol. Bioeng., 94(4):680-688 (2006)). Other antibody glycosylation variants are also included (See e.g., U.S. Pat. No. 6,602,684).

In some embodiments, an antibody provided herein has a dissociation constant (K_{D}) (e.g., affinity) of about 1 μM, 100 nM, 50 nM, 40 nM, 30 nM, 20 nM, 10 nM, 5 nM, 2 nM, 1 nM, 0.5 nM, 0.1 nM for the antibody target. In certain embodiments, the affinity is between about 0.1 nM to about 100 nM, from about 0.1 nM to about 50 nM, from about 0.1 nM to about 25 nM, from about 0.1 nM to about 10 nM, from about 0.1 nM to about 5 nM, from about 0.5 nM to about 5 nM, from about 0.5 nM to about 2 nM, or from about 0.5 nM to about 1 nM. The antibody target can be LIF. K_{D} can be measured by any suitable assay. In certain embodiments, K_{D} can be measured using surface plasmon resonance assays (e.g., using a BIACORE®-2000 or a BIACORE®-3000).

In some embodiments, one or more amino acid modifications may be introduced into the Fc region of an antibody provided herein, thereby generating an Fc region variant. An Fc region herein is a C-terminal region of an immunoglobulin heavy chain that contains at least a portion of the constant region. An Fc region includes native sequence Fc regions and variant Fc regions. The Fc region variant may comprise a human Fc region sequence (e.g., a human IgG1, IgG2, IgG3 or IgG4 Fc region) comprising an amino acid modification (e.g., a substitution) at one or more amino acid positions.

In some embodiments, the antibodies of this disclosure are variants that possess some but not all effector functions, which make it a desirable candidate for applications in which the half-life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. Non-limiting examples of in vitro assays to assess ADCC activity of a molecule of interest is described in U.S. Pat. Nos. 5,500,362 and 5,821,337. Alternatively, non-radioactive assays methods may be employed (e.g., ACTI™ and CytoTox 96® non-radioactive cytotoxicity assays). Useful effector cells for such assays include peripheral blood mononuclear cells (PBMC), monocytes, macrophages, and Natural Killer (NK) cells.

Antibodies can have increased half-lives and improved binding to the neonatal Fc receptor (FcRn) (See e.g., US 2005/0014934). Such antibodies can comprise an Fc region with one or more substitutions therein which improve binding of the Fc region to FcRn, and include those with substitutions at one or more of Fc region residues: 238, 256, 265, 272, 286, 303, 305, 307, 311, 312, 317, 340, 356, 360, 362, 376, 378, 380, 382, 413, 424 or 434 according to the EU numbering system (See e.g., U.S. Pat. No. 7,371,826). Other examples of Fc region variants are also contemplated (See e.g., Duncan & Winter, Nature 322:738-40 (1988); U.S. Pat. Nos. 5,648,260 and 5,624,821; and WO94/29351).

In some embodiments, it may be desirable to create cysteine engineered antibodies, e.g., “thioMAbs,” in which one or more residues of an antibody are substituted with cysteine residues. In some embodiments, the substituted residues occur at accessible sites of the antibody. Reactive thiol groups can be positioned at sites for conjugation to other moieties, such as drug moieties or linker drug moieties, to create an immunoconjugate. In some embodiments, any one or more of the following residues may be substituted with cysteine: V205 (Kabat numbering) of the light chain; A118 (EU numbering) of the heavy chain; and S400 (EU numbering) of the heavy chain Fc region.

In some embodiments, an antibody provided herein may be further modified to contain additional nonproteinaceous moieties that are known and available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n vinyl pyrrolidone)polyethylene glycol, polypropylene glycol homopolymers, polypropylen oxide/ethylene oxide co-polymers, polyoxyethylated polyols (e.g., glycerol), polyvinyl alcohol, and mixtures thereof. Polyethylene glycol propionaldehyde may have advantages in manufacturing due toits stability in water. The polymer may be of any molecular weight, and may be branched or unbranched. The number of polymers attached to the antibody may vary, and if two or more polymers are attached, they can be the same or different molecules.

The antibodies described herein can be encoded by a nucleic acid. A nucleic acid is a type of polynucleotide comprising two or more nucleotide bases. In certain embodiments, the nucleic acid is a component of a vector that can be used to transfer the polypeptide encoding polynucleotide into a cell. As used herein, the term “vector” refers to a nucleic acid molecule capable of transporting another nucleic acid to which it has been linked. One type of vector is a genomic integrated vector, or “integrated vector,” which can become integrated into the chromosomal DNA of the host cell. Another type of vector is an “episomal” vector, e.g., a nucleic acid capable of extra-chromosomal replication. Vectors capable of directing the expression of genes to which they are operatively linked are referred to herein as “expression vectors.” Suitable vectors comprise plasmids, bacterial artificial chromosomes, yeast artificial chromosomes, viral vectors and the like. In the expression vectors regulatory elements such as promoters, enhancers, polyadenylation signals for use in controlling transcription can be derived from mammalian, microbial, viral or insect genes. The ability to replicate in a host, usually conferred by an origin of replication, and a selection gene to facilitate recognition of transformants may additionally be incorporated. Vectors derived from viruses, such as lentiviruses, retroviruses, adenoviruses, adeno-associated viruses, and the like, may be employed. Plasmid vectors can be linearized for integration into a chromosomal location. Vectors can comprise sequences that direct site-specific integration into a defined location or restricted set of sites in the genome (e.g., AttP-AttB recombination). Additionally, vectors can comprise sequences derived from transposable elements.

The nucleic acids encoding the antibodies described herein can be used to infect, transfect, transform, or otherwise render a suitable cell transgenic for the nucleic acid, thus enabling the production of antibodies for commercial or therapeutic uses. Standard cell lines and methods for the production of antibodies from a large scale cell culture are known in the art. See e.g., Li et al., “Cell culture processes for monoclonal antibody production.” Mabs. 2010 September-October; 2(5): 466-477. In certain embodiments, the cell is a Eukaryotic cell. In certain embodiments, the Eukaryotic cell is a mammalian cell. In certain embodiments, the mammalian cell is a cell line useful for producing antibodies is a Chines Hamster Ovary cell (CHO) cell, an NS0 murine myeloma cell, or a PER.C6® cell. In certain embodiments, the nucleic acid encoding the antibody is integrated into a genomic locus of a cell useful for producing antibodies. In certain embodiments, described herein is a method of making an antibody comprising culturing a cell comprising a nucleic acid encoding an antibody under conditions in vitro sufficient to allow production and secretion of said antibody.

In certain embodiments, described herein, is a master cell bank comprising: (a) a mammalian cell line comprising a nucleic acid encoding an antibody described herein integrated at a genomic location; and (b) a cryoprotectant. In certain embodiments, the cryoprotectant comprises glycerol. In certain embodiments, the master cell bank comprises: (a) a CHO cell line comprising a nucleic acid encoding an antibody with (i) a heavy chain amino acid sequence set forth by any one of SEQ ID NOs: 1, 15, or 26; and (ii) a light chain amino acid sequence set forth by any one of SEQ ID NOs: 8, 20, 23, or 31 integrated at a genomic location; and (b) a cryoprotectant. In certain embodiments, the cryoprotectant comprises glycerol. In certain embodiments, the master cell bank is contained in a suitable vial able to withstand freezing by liquid nitrogen.

### LIF Binding Antibodies

The LIF antibodies described herein are monoclonal rabbit anti-LIF antibodies that have been humanized. In one aspect, described herein, is a recombinant antibody that specifically binds Leukemia Inhibitory Factor (LIF) comprising: (a) a heavy chain complementarity determining region 1 (VH-CDR1) comprising the amino acid sequence set forth in SEQ ID NO: 33 (TDYW); (b) a heavy chain complementarity determining region 2 (VH-CDR2) comprising the amino acid sequence set forth in an SEQ ID NO: 4 (IYVGTSGDT); (c) a heavy chain complementarity determining region 3 (VH-CDR3) comprising the amino acid sequence set forth in any one of SEQ ID NOs: 6 (ADNPYDYFNL) or 7 (AGADNPYDYFNLW); (d) a light chain complementarity determining region 1 (VL-CDR1) comprising the amino acid sequence set forth in SEQ ID NO: 9 (ETISGY); (e) a light chain complementarity determining region 2 (VL-CDR2) comprising the amino acid sequence set forth in SEQ ID NO: 11 (RAS); and (f) a light chain complementarity determining region 3 (VL-CDR3) comprising the amino acid sequence set forth in any one of SEQ ID NOs: 13 (QQGYSYS) or 14 (QQGYSYSDTDNV), wherein the recombinant antibody specifically binds to LIF.

### Clone 880

In another aspect, described herein, is a recombinant antibody that specifically binds Leukemia Inhibitory Factor (LIF) comprising: (a) a heavy chain complementarity determining region 1 (VH-CDR1) comprising the amino acid sequence set forth in any one of SEQ ID NOs: 2 (TDYWIC), 3 (GFSFSTDYWIC), or 33 (GFSFSTDY); (b) a heavy chain complementarity determining region 2 (VH-CDR2) comprising the amino acid sequence set forth in any one of SEQ ID NOs: 4 (IYVGTSGDT) or 5 (CIYVGTSGDTYYATWAKG); (c) a heavy chain complementarity determining region 3 (VH-CDR3) comprising the amino acid sequence set forth in any one of SEQ ID NOs: 6 (ADNPYDYFNL) or 7 (AGADNPYDYFNLW); (d) a light chain complementarity determining region 1 (VL-CDR1) comprising the amino acid sequence set forth in any one of SEQ ID NOs: 9 (ETISGY) or 10 (QASETISGYLS); (e) a light chain complementarity determining region 2 (VL-CDR2) comprising the amino acid sequence set forth in any one of SEQ ID NOs: 11 (RAS) or 12 (RASTLAS); and (f) a light chain complementarity determining region 3 (VL-CDR3) comprising the amino acid sequence set forth in any one of SEQ ID NOs: 13 (QQGYSYS) or 14 (QQGYSYSDTDNV), wherein the recombinant antibody specifically binds to LIF. In certain embodiments, the recombinant antibody comprises: (a) an immunoglobulin heavy chain variable region (VH) sequence with an amino acid sequence at least about 85%, 90%, 95%, 97%, 98%, 99%, or 100% identical to the amino acid sequence set forth in SEQ ID NO: 1; and (b) an immunoglobulin light chain variable region (VL) sequence with an amino acid sequence at least about 85%, 90%, 95%, 97%, 98%, 99%, or 100% identical to the amino acid sequence set forth in SEQ ID NO: 8.

### Clone 919

In another aspect, described herein, is a recombinant antibody that specifically binds Leukemia Inhibitory Factor (LIF) comprising (a) a heavy chain complementarity determining region 1 (VH-CDR1) comprising the amino acid sequence set forth in any one of SEQ ID NOs: 16 (TDYWWS), 17 (GFSFSTDYWW), or 18 (GFSFSTDYWWS); (b) a heavy chain complementarity determining region 2 (VH-CDR2) comprising the amino acid sequence set forth in any one of SEQ ID NOs: 4 (IYVGTSGDT) or 19 (YIYVGTSGDTYYNPSLKS); (c) a heavy chain complementarity determining region 3 (VH-CDR3) comprising the amino acid sequence set forth in any one of SEQ ID NOs: 6 (ADNPYDYFNL) or 7 (AGADNPYDYFNLW); (d) a light chain complementarity determining region 1 (VL-CDR1) comprising the amino acid sequence set forth in any one of SEQ ID NOs: 9 (ETISGY) or 21 (RASETISGYLN); (e) a light chain complementarity determining region 2 (VL-CDR2) comprising the amino acid sequence set forth in any one of SEQ ID NOs: 11 (RAS) or 22 (RASSLQS); and (f) a light chain complementarity determining region 3 (VL-CDR3) comprising the amino acid sequence set forth in any one of SEQ ID NOs: 13 (QQGYSYS) or 14 (QQGYSYSDTDNV), wherein the recombinant antibody specifically binds to LIF. In certain embodiments, the recombinant antibody that specifically binds Leukemia Inhibitory Factor (LIF) comprises: (a) an immunoglobulin heavy chain variable region (VH) sequence with an amino acid sequence at least about 85%, 90%, 95%, 97%, 98%, 99%, or 100% identical to the amino acid sequence set forth in SEQ ID NO: 15; and (b) an immunoglobulin light chain variable region (VL) sequence with an amino acid sequence at least about 85%, 90%, 95%, 97%, 98%, 99%, or 100% identical to the amino acid sequence set forth in SEQ ID NO: 20.

### Clone 920

In another aspect, described herein, is a recombinant antibody that specifically binds Leukemia Inhibitory Factor (LIF) comprising: (a) a heavy chain complementarity determining region 1 (VH-CDR1) comprising the amino acid sequence set forth in any one of SEQ ID NOs: 16 (TDYWWS), 17 (GFSFSTDYWW), or 18 (GFSFSTDYWWS); (b) a heavy chain complementarity determining region 2 (VH-CDR2) comprising the amino acid sequence set forth in any one of SEQ ID NOs: 4 (IYVGTSGDT) or 19 (YIYVGTSGDTYYNPSLKS); (c) a heavy chain complementarity determining region 3 (VH-CDR3) comprising the amino acid sequence set forth in any one of SEQ ID NOs: 6 (ADNPYDYFNL) or 7 (AGADNPYDYFNLW); (d) a light chain complementarity determining region 1 (VL-CDR1) comprising the amino acid sequence set forth in any one of SEQ ID NOs: 9 (ETISGY) or 24 (RASETISGYLA); (e) a light chain complementarity determining region 2 (VL-CDR2) comprising the amino acid sequence set forth in any one of SEQ ID NOs: 11 (RAS) or 25 (RASTLQS); and (f) a light chain complementarity determining region 3 (VL-CDR3) comprising the amino acid sequence set forth in any one of SEQ ID NOs: 13 (QQGYSYS) or 14 (QQGYSYSDTDNV), wherein the recombinant antibody specifically binds to LIF. In certain embodiments, the recombinant antibody that specifically binds Leukemia Inhibitory Factor (LIF) comprises: (a) an immunoglobulin heavy chain variable region (VH) sequence with an amino acid sequence at least about 85%, 90%, 95%, 97%, 98%, 99%, or 100% identical to the amino acid sequence set forth in SEQ ID NO: 15; and (b) an immunoglobulin light chain variable region (VL) sequence with an amino acid sequence at least about 85%, 90%, 95%, 97%, 98%, 99%, or 100% identical to the amino acid sequence set forth in SEQ ID NO: 23.

### Clone 937

In another aspect, described herein, is a recombinant antibody comprising: (a) a heavy chain complementarity determining region 1 (VH-CDR1) comprising the amino acid sequence set forth in any one of SEQ ID NOs: 27 (TDYWMS), 28 (GFSFSTDYWM), or 29 (GFSFSTDYWMS); (b) a heavy chain complementarity determining region 2 (VH-CDR2) comprising the amino acid sequence set forth in any one of SEQ ID NOs: 4 (IYVGTSGDT) or 30 (SIYVGTSGDTYYADSVKG); (c) a heavy chain complementarity determining region 3 (VH-CDR3) comprising the amino acid sequence set forth in any one of SEQ ID NOs: 6 (ADNPYDYFNL) or 7 (AGADNPYDYFNLW); (d) a light chain complementarity determining region 1 (VL-CDR1) comprising the amino acid sequence set forth in any one of SEQ ID NOs: 9 (ETISGY) or 24 (RASETISGYLA); (e) a light chain complementarity determining region 2 (VL-CDR2) comprising the amino acid sequence set forth in any one of SEQ ID NOs: 11 (RAS) or 32 (RASSLES); and (5) a light chain complementarity determining region 3 (VL-CDR3) comprising the amino acid sequence set forth in any one of SEQ ID NOs: 13 (QQGYSYS) or 14 (QQGYSYSDTDNV), wherein the recombinant antibody specifically binds to LIF. A recombinant antibody that specifically binds Leukemia Inhibitory Factor (LIF) comprises (a) an immunoglobulin heavy chain variable region (VH) sequence with an amino acid sequence at least about 85%, 90%, 95%, 97%, 98%, 99%, or 100% identical to the amino acid sequence set forth in SEQ ID NO: 26; and (b) an immunoglobulin light chain variable region (VL) sequence with an amino acid sequence at least about 85%, 90%, 95%, 97%, 98%, 99%, or 100% identical to the amino acid sequence set forth in SEQ ID NO: 31.

### Clone 938

In another aspect, described herein, is a recombinant antibody comprising: (a) a heavy chain complementarity determining region 1 (VH-CDR1) comprising the amino acid sequence set forth in any one of SEQ ID NOs: 27 (TDYWMS), 28 (GFSFSTDYWM), or 29 (GFSFSTDYWMS); (b) a heavy chain complementarity determining region 2 (VH-CDR2) comprising the amino acid sequence set forth in any one of SEQ ID NOs: 4 (IYVGTSGDT) or 30 (SIYVGTSGDTYYADSVKG); (c) a heavy chain complementarity determining region 3 (VH-CDR3) comprising the amino acid sequence set forth in any one of SEQ ID NOs: 6 (ADNPYDYFNL) or 7 (AGADNPYDYFNLW); (d) a light chain complementarity determining region 1 (VL-CDR1) comprising the amino acid sequence set forth in any one of SEQ ID NOs: 9 (ETISGY) or 21 (RASETISGYLN); (e) a light chain complementarity determining region 2 (VL-CDR2) comprising the amino acid sequence set forth in any one of SEQ ID NOs: 11 (RAS) or 22 (RASSLQS); and (f) a light chain complementarity determining region 3 (VL-CDR3) comprising the amino acid sequence set forth in any one of SEQ ID NOs: 13 (QQGYSYS) or 14 (QQGYSYSDTDNV), wherein the recombinant antibody specifically binds to LIF. In certain embodiments, the recombinant antibody recombinant antibody that specifically binds Leukemia Inhibitory Factor (LIF) comprises: (a) an immunoglobulin heavy chain variable region (VH) sequence with an amino acid sequence at least about 85%, 90%, 95%, 97%, 98%, 99%, or 100% identical to the amino acid sequence set forth in SEQ ID NO: 26; and (b) an immunoglobulin light chain variable region (VL) sequence with an amino acid sequence at least about 85%, 90%, 95%, 97%, 98%, 99%, or 100% identical to the amino acid sequence set forth in SEQ ID NO: 20.

### Therapeutic Indications

In certain embodiments, the antibodies disclosed herein inhibit LIF signaling in cells as determined by inhibition of phosphorylated STAT3 in response to LIF treatment. In certain embodiments, the IC₅₀ for biological inhibition of the antibody under serum starved conditions in U-251 cells is less than or equal to about 100, 75, 50, 40, 30, 20, 10, 5, or 1 nanomolar. In certain embodiments, the IC₅₀ for biological inhibition of the antibody under serum starved conditions in U-251 cells is less than or equal to about 900, 800, 700, 600, 500, 400, 300, 200, or 100 nanomolar.

In certain embodiments, the antibodies disclosed herein, are useful for treating tumors and cancers that express LIF. In certain embodiments, an individual treated with the antibodies of this disclosure has been selected for treatment as having a LIF positive tumor/cancer. In certain embodiments, the tumor is LIF positive or produces elevated levels of LIF. In certain embodiments, LIF positivity is determined in comparison to a reference value or a set pathological criteria. In certain embodiments, a LIF positive tumor expresses greater than 2-fold, 3- fold, 5-fold, 10-fold, 100-fold or more LIF than a non-transformed cell from which the tumor is derived. In certain embodiments, the tumor has acquired ectopic expression of LIF. A LIF positive tumor can be determined histologically using, for example, immunohistochemistry with an anti-LIF antibody; by commonly used molecular biology methods such as, for example, mRNA quantitation by real-time PCR or RNA-seq; or protein quantitation, for example, by western blot, flow cytometry, ELISA, or a homogenous protein quantitation assays (e.g., alphaLISA). In certain embodiments, the antibodies can be used to treat patients diagnosed with cancer. In certain embodiments, the cancer comprises one or more cancer stem cells or is one or more cancer stem cells. The polypeptide sequence of Human LIF is found at NCBI accession number NP 002300.1. While this sequence sets forth isoform one it will be recognized that certain naturally occurring variants of LIF with high sequence identity to that set forth by NCBI accession number NP 002300.1 can also be recognized by the antibodies descried herein.

In certain embodiments, the antibodies disclosed herein, are useful for treating tumors in cancers that express the LIF receptor (CD118). A LIF receptor positive tumor can be determined by histopathology or flow cytometry, and, in certain embodiments, comprises a cell that binds a LIF receptor antibody greater than 2×, 3×, 3×, 4×, 5×, 10× or more than an isotype control. In certain embodiments, the tumor has acquired ectopic expression of the LIF receptor. In a certain embodiment, the cancer is a cancer stem cell. In a certain embodiment, a LIF positive tumor or cancer can be determined by immunohistochemistry using anti-LIF an anti-LIF antibody. In a certain embodiment, a LIF positive tumor is determined by IHC analysis with a LIF Level in the top 10%, 20%, 30%, 40%, or top 50% of tumors.

In certain embodiments, disclosed herein, are antibodies useful for the treatment of a cancer or tumor. In certain embodiments, the cancer comprises breast, heart, lung, small intestine, colon, spleen, kidney, bladder, head, neck, ovarian, prostate, brain, pancreatic, skin, bone, bone marrow, blood, thymus, uterine, testicular, and liver tumors. In certain embodiments, tumors which can be treated with the antibodies of the invention comprise adenoma, adenocarcinoma, angiosarcoma, astrocytoma, epithelial carcinoma, germinoma, glioblastoma, glioma, hemangioendothelioma, hemangiosarcoma, hematoma, hepatoblastoma, leukemia, lymphoma, medulloblastoma, melanoma, neuroblastoma, osteosarcoma, retinoblastoma, rhabdomyosarcoma, sarcoma and/or teratoma. In certain embodiments, the tumor/cancer is selected from the group of acral lentiginous melanoma, actinic keratosis, adenocarcinoma, adenoid cystic carcinoma, adenomas, adenosarcoma, adenosquamous carcinoma, astrocytic tumors, Bartholin gland carcinoma, basal cell carcinoma, bronchial gland carcinoma, capillary carcinoid, carcinoma, carcinosarcoma, cholangiocarcinoma, chondrosarcoma, cystadenoma, endodermal sinus tumor, endometrial hyperplasia, endometrial stromal sarcoma, endometrioid adenocarcinoma, ependymal sarcoma, Swing's sarcoma, focal nodular hyperplasia, gastronoma, germ line tumors, glioblastoma, glucagonoma, hemangioblastoma, hemangioendothelioma, hemangioma, hepatic adenoma, hepatic adenomatosis, hepatocellular carcinoma, insulinite, intraepithelial neoplasia, intraepithelial squamous cell neoplasia, invasive squamous cell carcinoma, large cell carcinoma, liposarcoma, lung carcinoma, lymphoblastic leukemia, lymphocytic leukemia, leiomyosarcoma, melanoma, malignant melanoma, malignant mesothelial tumor, nerve sheath tumor, medulloblastoma, medulloepithelioma, mesothelioma, mucoepidermoid carcinoma, myeloid leukemia, neuroblastoma, neuroepithelial adenocarcinoma, nodular melanoma, osteosarcoma, ovarian carcinoma, papillary serous adenocarcinoma, pituitary tumors, plasmacytoma, pseudosarcoma, prostate carcinoma, pulmonary blastoma, renal cell carcinoma, retinoblastoma, rhabdomyosarcoma, sarcoma, serous carcinoma, squamous cell carcinoma, small cell carcinoma, soft tissue carcinoma, somatostatin secreting tumor, squamous carcinoma, squamous cell carcinoma, undifferentiated carcinoma, uveal melanoma, verrucous carcinoma, vagina/vulva carcinoma, VIPpoma, and Wilm's tumor. In certain embodiments, the tumor/cancer to be treated with one or more antibodies of the invention comprise brain cancer, head and neck cancer, colorectal carcinoma, acute myeloid leukemia, pre-B-cell acute lymphoblastic leukemia, bladder cancer, astrocytoma, preferably grade II, III or IV astrocytoma, glioblastoma, glioblastoma multiforme, small cell cancer, and non-small cell cancer, preferably non-small cell lung cancer, lung adenocarcinoma, metastatic melanoma, androgen-independent metastatic prostate cancer, androgen-dependent metastatic prostate cancer, prostate adenocarcinoma, and breast cancer, preferably breast ductal cancer, and/or breast carcinoma. In certain embodiments, the cancer treated with the antibodies of this disclosure comprises glioblastoma. In certain embodiments, the cancer treated with one or more antibodies of this disclosure comprises pancreatic cancer. In certain embodiments, the cancer treated with one or more antibodies of this disclosure comprises ovarian cancer. In certain embodiments, the cancer treated with one or more antibodies of this disclosure comprises lung cancer. In certain embodiments, the cancer treated with one or more antibodies of this disclosure comprises prostate cancer. In certain embodiments, the cancer treated with one or more antibodies of this disclosure comprises colon cancer. In certain embodiments, the cancer treated comprises glioblastoma, pancreatic cancer, ovarian cancer, colon cancer, prostate cancer, or lung cancer. In a certain embodiment, the cancer is refractory to other treatment. In a certain embodiment, the cancer treated is relapsed. In a certain embodiment, the cancer is a relapsed/refractory glioblastoma, pancreatic cancer, ovarian cancer, colon cancer, prostate cancer, or lung cancer.

### Therapeutic Methods

In certain embodiments, the antibodies can be administered to a subject in need thereof by any route suitable for the administration of antibody-containing pharmaceutical compositions, such as, for example, subcutaneous, intraperitoneal, intravenous, intramuscular, intratumoral, or intracerebral, etc. In certain embodiments, the antibodies are administered intravenously. In certain embodiments, the antibodies are administered on a suitable dosage schedule, for example, weekly, twice weekly, monthly, twice monthly, once every two weeks, once every three weeks, or once a month etc. In certain embodiments, the antibodies are administered once every three weeks. The antibodies can be administered in any therapeutically effective amount. In certain embodiments, the therapeutically acceptable amount is between about 0.1 mg/kg and about 50 mg/kg. In certain embodiments, the therapeutically acceptable amount is between about 1 mg/kg and about 40 mg/kg. In certain embodiments, the therapeutically acceptable amount is between about 5 mg/kg and about 30 mg/kg.

### Pharmaceutically Acceptable Excipients, Carriers, and Diluents

In certain embodiments, the anti-LIF antibodies of the current disclosure are included in a pharmaceutical composition comprising one or more pharmaceutically acceptable excipients, carriers, or diluents. In certain embodiments, the antibodies of the current disclosure are administered suspended in a sterile solution. In certain embodiments, the solution comprises about 0.9% NaCl. In certain embodiments, the solution comprises about 5% glucose or dextrose. In certain embodiments, the solution further comprises one or more of: buffers, for example, acetate, citrate, histidine, succinate, phosphate, bicarbonate and hydroxymethylaminomethane (Tris); surfactants, for example, polysorbate 80 (Tween 80), polysorbate 20 (Tween 20), and poloxamer 188; polyol/disaccharide/polysaccharides, for example, glucose, dextrose, mannose, mannitol, sorbitol, sucrose, trehalose, and dextran 40; amino acids, for example, glycine, histidine, or arginine; antioxidants, for example, ascorbic acid, methionine; or chelating agents, for example, EGTA or EGTA. In certain embodiments, the antibodies of the current disclosure are shipped/stored lyophilized and reconstituted before administration. In certain embodiments, lyophilized antibody formulations comprise a bulking agent such as, mannitol, sorbitol, sucrose, trehalose, and dextran 40. The lyophilized formulation can be contained in a vial comprised of glass. The antibodies when formulated, whether reconstituted or not, can be buffered at a certain pH, generally less than 7.0. In certain embodiments, the pH can be between 4.5 and 6.5, 4.5 and 6.0, 4.5 and 5.5, 4.5 and 5.0, or 5.0 and 6.0.

In certain embodiments, described herein is a method of preparing a cancer treatment comprising admixing one or more pharmaceutically acceptable excipients, carriers, or diluents and an antibody of the current disclosure. In certain embodiments, described herein is a method of preparing a cancer treatment for storage or shipping comprising lyophilizing one or more antibodies of the current disclosure.

### EXAMPLES

The following illustrative examples are representative of embodiments of the compositions and methods described herein and are not meant to be limiting in any way.

### Example 1— Generation of a LIF Binding Antibody

In an effort to identify antibodies that block the LIF:LIFR interaction, we embarked on an immunization campaign in rabbits. From this campaign, a monoclonal antibody (mAb) clone B7, which blocks the human LIF:LIFR interaction with high affinity was discovered. The methods and results below describe the screening to identify B7 as well as the functional characterization of mAb B7 and its humanized variants.

Two rabbits were immunized with recombinant human LIF. One week after the third boost, blood was collected and the serum titers were tested to confirm immune responses. The rabbit with the highest titer was given a final boost, and blood was collected. A yeast display library was generated using amplified V regions from PBMC's isolated from this immunized rabbit, and the library was then FACS sorted against fluorescently labelled recombinant human LIF. 96 clones were screened for binding (Table 1) and competition (Table 2) using a commercial antibody known to inhibit LIF:LIFR binding. From this screen, clone B7 was identified, which blocks human LIF:LIFR but does not cross-react to mouse LIF.

TABLE 1
Summary of ELISA results from the binding screen of 96 FACS sorted clones to human
LIF.

| OD450 | 1 | 2 | 3 | 4 | 5 | 6 | 7 | 8 | 9 | 10 | 11 | 12 |
|---|---|---|---|---|---|---|---|---|---|---|---|---|
| A | 0.92 | 0.31 | 0.53 | 1.75 | 1.84 | 1.39 | 1.99 | 2.24 | 1.19 | 1.92 | 0.77 | 1.20 |
| B | 1.89 | 0.53 | 0.33 | 2.04 | 1.91 | 2.23 | 2.43 | 2.27 | 1.08 | 1.49 | 1.30 | 1.83 |
| C | 1.47 | 0.82 | 2.05 | 1.66 | 1.70 | 2.46 | 2.10 | 1.89 | 2.14 | 0.90 | 1.89 | 1.71 |
| D | 1.85 | 1.74 | 2.28 | 0.30 | 2.30 | 1.45 | 2.27 | 2.14 | 1.51 | 1.51 | 2.09 | 0.84 |
| E | 1.65 | 1.42 | 1.52 | 0.34 | 0.35 | 1.92 | 0.98 | 1.50 | 1.75 | 2.07 | 2.34 | 0.17 |
| F | 0.04 | 1.25 | 1.45 | 1.37 | 0.04 | 1.07 | 0.51 | 1.88 | 2.04 | 1.02 | 1.56 | 2.01 |
| G | 1.67 | 1.45 | 0.90 | 2.29 | 1.60 | 1.78 | 1.64 | 2.07 | 2.46 | 2.12 | 0.35 | 1.85 |
| H | 0.82 | 1.41 | 1.23 | 1.67 | 1.36 | 1.13 | 1.73 | 1.48 | 1.50 | 1.38 | 0.42 | 0.05 |

TABLE 2
Summary of ELISA results from a competition screen with a known LIF:LIFR blocking antibody
and identification of clone B7 as a blocking IgG.

| OD450 | 1 | 2 | 3 | 4 | 5 | 6 | 7 | 8 | 9 | 10 | 11 | 12 |
|---|---|---|---|---|---|---|---|---|---|---|---|---|
| A | 0.072 | 0.372 | 0.640 | 1.928 | 1.857 | 1.378 | 0.314 | 0.229 | 1.214 | 1.886 | 0.434 | 1.004 |
| B | 2.074 | 0.636 | 0.440 | 0.173 | 1.846 | 0.177 | 0.142 | 1.908 | 1.508 | 1.584 | 1.638 | 0.839 |
| C | 0.075 | 1.166 | 0.599 | 1.801 | 0.930 | 0.137 | 1.781 | 1.630 | 0.892 | 1.360 | 1.867 | 1.051 |
| D | 1.802 | 1.312 | 0.137 | 0.795 | 0.098 | 1.574 | 0.186 | 2.020 | 1.500 | 0.940 | 1.752 | 1.010 |
| E | 1.855 | 0.079 | 0.833 | 0.367 | 0.848 | 0.238 | 1.134 | 1.664 | 1.745 | 1.967 | 1.933 | 0.228 |
| F | 0.101 | 1.389 | 0.198 | 1.335 | 0.051 | 1.242 | 0.544 | 1.824 | 0.142 | 1.861 | 1.561 | 1.353 |
| G | 1.481 | 1.354 | 0.483 | 0.152 | 0.123 | 1.664 | 1.556 | 1.754 | 0.139 | 1.750 | 0.374 | 0.085 |
| H | 0.382 | 1.193 | 1.303 | 0.162 | 1.361 | 1.519 | 0.094 | 0.103 | 1.345 | 1.504 | 0.311 | 0.060 |

### Example 2— Generation and Testing of Humanized Versions of Clone B7

Clone B7 was sequenced and its heavy and light chain V regions were identified. The light chain variable region was mutated in position 80 from cysteine to proline to avoid generation of a free cysteine in full antibody constructs that possess a constant region. The resulting V regions were then grafted onto a human IgG1 heavy chain constant region and a human kappa constant region to generate a rabbit/human chimeric mAb, resulting in the clone NB0880. Humanized variants were also generated by CDR grafting, where the rabbit mAb B7 V regions were blasted against the IMGT database and the closest related VH and VL human germline framework regions were selected as templates for CDR grafting. CDRs were identified based on the IMGT numbering scheme. In total, 15 humanized variants were generated and tested for binding to LIF by ELISA (FIG. 1). Four antibodies (NB0919, NB0920, NB0937 and NB0938) were selected for further testing and their binding and functional properties were assessed and are summarized in Table 3.

### Binding EC50s

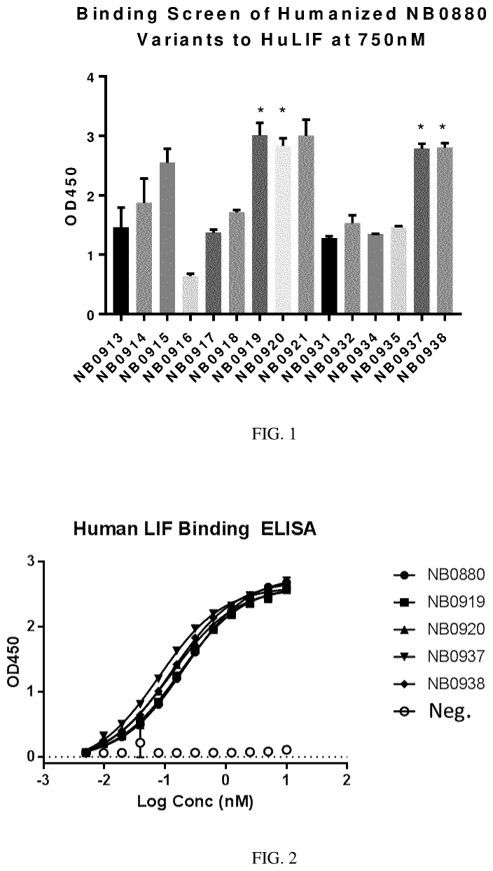

Recombinant human LIF was coated on maxisorp plates overnight at 4° C. The next day, plates were blocked with casein blocking buffer for 1 hour at 37° C. Titrations of each antibody were added to the plates and allowed to bind for 1 hour at room temperature. Plates were washed 4× with PBST followed by incubation with an HRP conjugated anti-human Fc secondary for 30 minutes at RT. Plates were then washed again with 4×PBST and then developed using TMB substrate and 1M HCl. Binding EC50 curves are shown in FIG. 2 and the respective values are listed in Table 3.

### Affinity Measurements

Affinities were determined using biolayer interferometry on the Octet platform. Anti-human Fc biosensors were used to load chimeric B7 and humanized variants, and titrations of recombinant human LIF (200 nM→0 nM) were associated for 180 seconds followed by a 300 second dissociation step. Affinity measurements are recorded in Table 3.

### Recombinant LIF:LIFR Blocking Assay

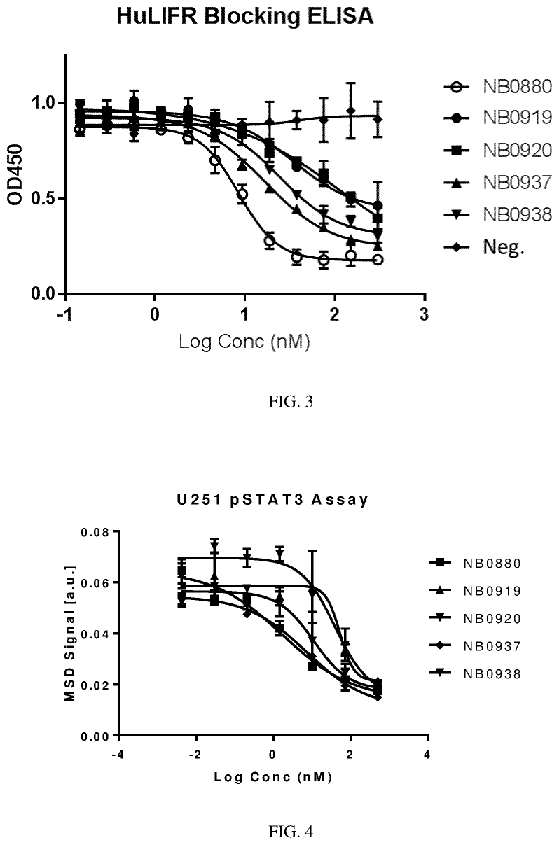

Recombinant human LIFR was coated on maxisorp plates overnight at 4 C. The next day, plates were blocked with casein blocking buffer for 1 hr at 37 C. During this period, titrations of each antibody were mixed with biotinylated recombinant human LIF. Following blocking, the antibody/antigen premix was added to the plates and allowed to incubate for 1 hr at RT. Plates were then washed with 4×PBST and an HRP conjugated avidin secondary reagent was added for 30 min at room temperature. Plates were washed again with 4×PBST and then developed using TMB substrate and 1M HCl. Blocking IC50 curves are shown in FIG. 3 and the respective values are listed in Table 3.

pSTAT3 Inhibition Assay

U251 cells were cultured on tissue culture treated plates for 48 hours, followed by treatment with recombinant human LIF that was pre-mixed with titrations of each antibody. After 10 minutes of treatment, cells were lysed and lysates were added to plates that either measure total STAT3 or pSTAT3. Plates were analyzed on the MSD platform and normalized ratios of pSTAT3/total STAT3 were plotted to obtain dose response curves for each antibody. Blocking IC50 curves are shown in FIG. 4 and the respective values are listed in Table 3.

TABLE 3
Summary of binding characteristics of humanized antibody clones

| | NB0880 | NB0919 | NB0920 | NB0937 | NB0938 |
|---|---|---|---|---|---|
| Binding EC50 (nM) | 0.21 | 0.17 | 0.12 | 0.08 | 0.13 |
| LIF:LIFR Blocking | 8.61 | ~ | ~ | 16.58 | 24.42 |
| Assay IC50 (nM) | | | | | |
| pSTAT3 inhibition | 1.74 | 52.69 | 38.68 | 7.22 | 10.96 |
| assay IC50 (nM) | | | | | |
| Affinity (nM) | 0.69 | 19.6 | 9.97 | 5.02 | 10.6 |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

SEQUENCES DISCLOSED HEREIN

| SEQ | |
|---|---|
| ID | Sequence |
| 1 | QSLEESGGDLVKPEGSLTLTCTASGFSFSTD |
| | YWICWVRQAPGKGLEWIACIYVGTSGDTYYA |
| | TWAKGRFTISKTSSTTVTLQMTSLTAADTAT |
| | YFCAGADNPYDYFNLWGPGTLVTVSS |
| 2 | TDYWIC |
| 3 | GFSFSTDYWIC |
| 4 | IYVGTSGDT |
| 5 | CIYVGTSGDTYYATWAKG |
| 6 | ADNPYDYFNL |
| 7 | AGADNPYDYFNLW |
| 8 | GTPDMTQTPASMEVAVGGTVTIKCQASETISGY |
| | LSWYQQKPGQRPKLLMYRASTLASGVSSRFKGS |
| | GSGTQFTLTISGVECADAATYYCQQGYSYSDTD |
| | NVFGGGTEVV |
| 9 | ETISGY |
| 10 | QASETISGYLS |
| 11 | RAS |
| 12 | RASTLAS |
| 13 | QQGYSYS |
| 14 | QQGYSYSDTDNV |
| 15 | QVQLQESGPGLVKPSDTLSLTCTVSGFSFSTDY |
| | WWSWIRQPPGKGLEWIGYIYVGTSGDTYYNPSL |
| | KSRVTISVDTSKNQFSLKLSSVTAADTAVYYCA |
| | GADNPYDYFNLWGQGTLVTVSS |
| 16 | TDYWWS |
| 17 | GFSFSTDYWW |
| 18 | GFSFSTDYWWS |
| 19 | YIYVGTSGDTYYNPSLKS |
| 20 | DIQMTQSPSSLSASVGDRVTITCRASETISGYL |
| | NWYQQKPGKAPKLLIYRASSLQSGVPSRFSGSG |
| | SGTDFTLTISSLQPEDFATYYCQQGYSYSDTDN |
| | VFGGGTKVEIK |
| 21 | RASETISGYLN |
| 22 | RASSLQS |
| 23 | DIQMTQSPSSLSASVGDRVTITCRASETISGYLA |
| | WYQQKPGKVPKLLIYRASTLQSGVPSRFSGSGSG |
| | TDFTLTISSLQPEDVATYYCQQGYSYSDTDNVFG |
| | GGTKVEIK |
| 24 | RASETISGYLA |
| 25 | RASTLQS |
| 26 | EVQLVESGGGLVQPGGSLRLSCAASGFSFSTDYW |
| | MSWVRQAPGKGLEWVSSIYVGTSGDTYYADSVKG |
| | RFTISRDNSKNTLYLQMDSLRAEDTAIYYCAGAD |
| | NPYDYFNLWGQGTLVTVS |
| 27 | TDYWMS |
| 28 | GFSFSTDYWM |
| 29 | GFSFSTDYWMS |
| 30 | SIYVGTSGDTYYADSVKG |
| | DIQMTQSPSSVSASVGDRVTITCRASETISGYLA |
| | WYQQKPGKAPKLLIYRASSLESGVPSRFSGSGSG |
| | TEFTLTISSLQPDDFATYYCQQGYSYSDTDNVFG |
| | GGTKVEIK |
| 32 | RASSLES |
| 33 | GFSFSTDY |

What is claimed is:

1. A recombinant antibody that specifically binds Leukemia Inhibitory Factor (LIF) comprising:
a) a heavy chain complementarity determining region 1 (VH-CDR1) comprising the amino acid sequence set forth in any one of SEQ ID NOs: 2; (TDYWIC) 3; (GFSFSTDYWIC); or 33 (GFSFSTDY);
b) a heavy chain complementarity determining region 2 (VH-CDR2) comprising the amino acid sequence set forth in any one of SEQ ID NOs: 4 (IYVGTSGDT) or 5 (CIYVGTSGDTYYATWAKG);
c) a heavy chain complementarity determining region 3 (VH-CDR3) comprising the amino acid sequence set forth in any one of SEQ ID NOs: 6 (ADNPYDYFNL) or 7 (AGADNPYDYFNLW);
d) a light chain complementarity determining region 1 (VL-CDR1) comprising the amino acid sequence set forth in any one of SEQ ID NOs: 9 (ETISGY) or 10 (QASETISGYLS);
e) a light chain complementarity determining region 2 (VL-CDR2) comprising the amino acid sequence set forth in any one of SEQ ID NOs: 11 (RAS) or 12 (RASTLAS); and
f) a light chain complementarity determining region 3 (VL-CDR3) comprising the amino acid sequence set forth in any one of SEQ ID NOs: 13 (QQGYSYS) or 14 (QQGYSYSDTDNV).

2. A recombinant antibody that specifically binds Leukemia Inhibitory Factor (LIF) comprising:
a) a heavy chain complementarity determining region 1 (VH-CDR1) comprising the amino acid sequence set forth in any one of SEQ ID NOs: 16 (TDYWWS); 17 (GFSFSTDYWW); or 18 (GFSFSTDYWWS);
b) a heavy chain complementarity determining region 2 (VH-CDR2) comprising the amino acid sequence set forth in any one of SEQ ID NOs: 4 (IYVGTSGDT) or 19 (YIYVGTSGDTYYNPSLKS);
c) a heavy chain complementarity determining region 3 (VH-CDR3) comprising the amino acid sequence set forth in any one of SEQ ID NOs: 6 (ADNPYDYFNL) or 7 (AGADNPYDYFNLW);
d) a light chain complementarity determining region 1 (VL-CDR1) comprising the amino acid sequence set forth in any one of SEQ ID NOs: 9 (ETISGY) or 21 (RASETISGYLN);
e) a light chain complementarity determining region 2 (VL-CDR2) comprising the amino acid sequence set forth in any one of SEQ ID NOs: 11 (RAS) or 22 (RASSLQS); and
f) a light chain complementarity determining region 3 (VL-CDR3) comprising the amino acid sequence set forth in any one of SEQ ID NOs: 13 (QQGYSYS) or 14 (QQGYSYSDTDNV).

3. A recombinant antibody that specifically binds Leukemia Inhibitory Factor (LIF) comprising:
a) a heavy chain complementarity determining region 1 (VH-CDR1) comprising the amino acid sequence set forth in any one of SEQ ID NOs: 16 (TDYWWS); 17 (GFSFSTDYWW); or 18 (GFSFSTDYWWS);
b) a heavy chain complementarity determining region 2 (VH-CDR2) comprising the amino acid sequence set forth in any one of SEQ ID NOs: 4 (IYVGTSGDT) or 19 (YIYVGTSGDTYYNPSLKS);
c) a heavy chain complementarity determining region 3 (VH-CDR3) comprising the amino acid sequence set forth in any one of SEQ ID NOs: 6 (ADNPYDYFNL) or 7 (AGADNPYDYFNLW);
d) a light chain complementarity determining region 1 (VL-CDR1) comprising the amino acid sequence set forth in any one of SEQ ID NOs: 9 (ETISGY) or 24 (RASETISGYLA);
e) a light chain complementarity determining region 2 (VL-CDR2) comprising the amino acid sequence set forth in any one of SEQ ID NOs: 11 (RAS) or 25 (RASTLQS); and
f) a light chain complementarity determining region 3 (VL-CDR3) comprising the amino acid sequence set forth in any one of SEQ ID NOs: 13 (QQGYSYS) or 14 (QQGYSYSDTDNV).

4. A recombinant antibody that specifically binds Leukemia Inhibitory Factor (LIF) comprising:
a) a heavy chain complementarity determining region 1 (VH-CDR1) comprising the amino acid sequence set forth in any one of SEQ ID NOs: 27 (TDYWMS); 28 (GFSFSTDYWM); or 29 (GFSFSTDYWMS);
b) a heavy chain complementarity determining region 2 (VH-CDR2) comprising the amino acid sequence set forth in any one of SEQ ID NOs: 4 (IYVGTSGDT) or 30 (SIYVGTSGDTYYADSVKG);
c) a heavy chain complementarity determining region 3 (VH-CDR3) comprising the amino acid sequence set forth in any one of SEQ ID NOs: 6 (ADNPYDYFNL) or 7 (AGADNPYDYFNLW);
d) a light chain complementarity determining region 1 (VL-CDR1) comprising the amino acid sequence set forth in any one of SEQ ID NOs: 9 (ETISGY) or 24 (RASETISGYLA);
e) a light chain complementarity determining region 2 (VL-CDR2) comprising the amino acid sequence set forth in any one of SEQ ID NOs: 11 (RAS) or 32 (RASSLES); and
f) a light chain complementarity determining region 3 (VL-CDR3) comprising the amino acid sequence set forth in any one of SEQ ID NOs: 13 (QQGYSYS) or 14 (QQGYSYSDTDNV).

5. A recombinant antibody that specifically binds Leukemia Inhibitory Factor (LIF) comprising:
a) a heavy chain complementarity determining region 1 (VH-CDR1) comprising the amino acid sequence set forth in any one of SEQ ID NOs: 27 (TDYWMS); 28 (GFSFSTDYWM); or 29 (GFSFSTDYWMS);
b) a heavy chain complementarity determining region 2 (VH-CDR2) comprising the amino acid sequence set forth in any one of SEQ ID NOs: 4 (IYVGTSGDT) or 30 (SIYVGTSGDTYYADSVKG);
c) a heavy chain complementarity determining region 3 (VH-CDR3) comprising the amino acid sequence set forth in any one of SEQ ID NOs: 6 (ADNPYDYFNL) or 7 (AGADNPYDYFNLW);
d) a light chain complementarity determining region 1 (VL-CDR1) comprising the amino acid sequence set forth in any one of SEQ ID NOs: 9 (ETISGY) or 21 (RASETISGYLN);
e) a light chain complementarity determining region 2 (VL-CDR2) comprising the amino acid sequence set forth in any one of SEQ ID NOs: 11 (RAS) or 22 (RASSLQS); and
f) a light chain complementarity determining region 3 (VL-CDR3) comprising the amino acid sequence set forth in any one of SEQ ID NOs: 13 (QQGYSYS) or 14 (QQGYSYSDTDNV).

6. A recombinant antibody that specifically binds Leukemia Inhibitory Factor (LIF) comprising:
a) a heavy chain complementarity determining region 1 (VH-CDR1) comprising the amino acid sequence set forth in SEQ ID NO: 33 (TDYW);
b) a heavy chain complementarity determining region 2 (VH-CDR2) comprising the amino acid sequence set forth in an SEQ ID NO: 4 (IYVGTSGDT);
c) a heavy chain complementarity determining region 3 (VH-CDR3) comprising the amino acid sequence set forth in any one of SEQ ID NOs: 6 (ADNPYDYFNL) or 7 (AGADNPYDYFNLW);
d) a light chain complementarity determining region 1 (VL-CDR1) comprising the amino acid sequence set forth in SEQ ID NO: 9 (ETISGY);
e) a light chain complementarity determining region 2 (VL-CDR2) comprising the amino acid sequence set forth in SEQ ID NO: 11 (RAS); and
f) a light chain complementarity determining region 3 (VL-CDR3) comprising the amino acid sequence set forth in any one of SEQ ID NOs: 13 (QQGYSYS) or 14 (QQGYSYSDTDNV).

7. The recombinant antibody of any one of claims 1 to 6, wherein the recombinant antibody is chimeric or humanized.

8. The recombinant antibody of any one of claims 1 to 6, wherein the recombinant antibody is an IgG antibody.

9. The recombinant antibody of any one of claims 1 to 6, wherein the recombinant antibody comprises a Fab, F(ab)₂, a single-domain antibody, a single chain variable fragment (scFv), or a nanobody.

10. A recombinant antibody that specifically binds Leukemia Inhibitory Factor (LIF) comprising:
a) an immunoglobulin heavy chain variable region (VH) sequence with an amino acid sequence at least about 85%, 90%, 95%, 97%, 98%, 99%, or 100% identical to the amino acid sequence set forth in SEQ ID NO: 1; and
b) an immunoglobulin light chain variable region (VL) sequence with an amino acid sequence at least about 85%, 90%, 95%, 97%, 98%, 99%, or 100% identical to the amino acid sequence set forth in SEQ ID NO: 8.

11. A recombinant antibody that specifically binds Leukemia Inhibitory Factor (LIF) comprising:
a) an immunoglobulin heavy chain variable region (VH) sequence with an amino acid sequence at least about 85%, 90%, 95%, 97%, 98%, 99%, or 100% identical to the amino acid sequence set forth in SEQ ID NO: 15; and
b) an immunoglobulin light chain variable region (VL) sequence with an amino acid sequence at least about 85%, 90%, 95%, 97%, 98%, 99%, or 100% identical to the amino acid sequence set forth in SEQ ID NO: 20.

12. A recombinant antibody that specifically binds Leukemia Inhibitory Factor (LIF) comprising:
a) an immunoglobulin heavy chain variable region (VH) sequence with an amino acid sequence at least about 85%, 90%, 95%, 97%, 98%, 99%, or 100% identical to the amino acid sequence set forth in SEQ ID NO: 15; and
b) an immunoglobulin light chain variable region (VL) sequence with an amino acid sequence at least about 85%, 90%, 95%, 97%, 98%, 99%, or 100% identical to the amino acid sequence set forth in SEQ ID NO: 23.

13. A recombinant antibody that specifically binds Leukemia Inhibitory Factor (LIF) comprising:
a) an immunoglobulin heavy chain variable region (VH) sequence with an amino acid sequence at least about 85%, 90%, 95%, 97%, 98%, 99%, or 100% identical to the amino acid sequence set forth in SEQ ID NO: 26; and
b) an immunoglobulin light chain variable region (VL) sequence with an amino acid sequence at least about 85%, 90%, 95%, 97%, 98%, 99%, or 100% identical to the amino acid sequence set forth in SEQ ID NO: 31.

14. A recombinant antibody that specifically binds Leukemia Inhibitory Factor (LIF) comprising:
a) an immunoglobulin heavy chain variable region (VH) sequence with an amino acid sequence at least about 85%, 90%, 95%, 97%, 98%, 99%, or 100% identical to the amino acid sequence set forth in SEQ ID NO: 26; and
b) an immunoglobulin light chain variable region (VL) sequence with an amino acid sequence at least about 85%, 90%, 95%, 97%, 98%, 99%, or 100% identical to the amino acid sequence set forth in SEQ ID NO: 20.

15. The recombinant antibody of any one of claims 10 to 14, wherein the recombinant antibody is chimeric or humanized.

16. The recombinant antibody of any one of claims 10 to 14, wherein the recombinant antibody is an IgG antibody.

17. The recombinant antibody of any one of claims 10 to 14, wherein the recombinant antibody comprises a Fab, F(ab)₂, a single-domain antibody, a single chain variable fragment (scFv), or a nanobody.

18. A pharmaceutical composition comprising the recombinant antibody of any one of claims 1 to 17 and a pharmaceutically acceptable excipient, carrier, or diluent.

19. The pharmaceutical composition of claim 18, formulated for intravenous administration.

20. The recombinant antibody of any one of claims 1 to 17 or the pharmaceutical composition of claim 18 or 19 for use in treating cancer.

21. The recombinant antibody of claim 20, wherein the cancer comprises glioblastoma, pancreatic cancer, ovarian cancer, colon cancer, prostate cancer, or lung cancer.

22. A method of treating a cancer in an individual comprising administering an effective amount of the recombinant antibody of any one of claims 1 to 17 or the pharmaceutical composition of claim 18 or 19 to an individual diagnosed with or suspected of being afflicted with cancer.

23. The method of claim 22, wherein the cancer comprises glioblastoma, pancreatic cancer, ovarian cancer, colon cancer, prostate cancer, or lung cancer.

24. A nucleic acid encoding the recombinant antibody of any one of claims 1 to 17.

25. A cell comprising the nucleic acid of claim 24.

26. The cell of claim 25, wherein the cell is a eukaryotic cell.

27. The cell of claim 25, wherein the cell is a Chinese Hamster Ovary (CHO) cell.

28. A method of making a cancer treatment comprising incubating the cell of any one of claims 24 to 27 in a culture medium under conditions sufficient to secrete the recombinant antibody of any one of claims 1 to 17 into the culture medium.

29. The method of claim 28, further comprising subjecting the culture medium to at least one purification step.

30. A method of making a cancer treatment comprising admixing the recombinant antibody of any one of claims 1 to 17 and a pharmaceutically acceptable excipient, carrier, or diluent.
